(19) United States
(12) Patent Application Publication
(10) Pub. No.: US 2009/0274757 A1
(43) Pub. Date: Nov. 5, 2009

(54) PHARMACEUTICAL POLISH FORMULATIONS

(75) Inventor: John C. CLARK, Peru, NY (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 12/433,585

(22) Filed: Apr. 30, 2009

Related U.S. Application Data
(60) Provisional application No. 61/049,539, filed on May 1, 2008.

(51) Int. Cl. A61K 9/42; C11D 7/60; A61K 9/00; A61K 31/565

(52) U.S. Cl. 424/476; 106/5; 106/10; 106/3; 424/400; 514/182; 514/170

(57) ABSTRACT

Polish compositions for coating pharmaceutical solid dosage forms such as tablets are disclosed. The polish composition comprises water, coating agent, and a film forming agent. Polished pharmaceutical solid dosage forms such as tablets comprising a polished exterior surface are also disclosed. Processes of polishing pharmaceutical solid dosage forms such as tablets are disclosed.

This application claims benefit of priority to U.S. provisional patent application Ser. No. 61/049,539 filed May 1, 2008, which is hereby incorporated by reference in its entirety.

### FIELD OF THE INVENTION

The present invention relates, inter alia, to new pharmaceutical polish formulations which provide luster and slip to coated pharmaceutical solid dosage forms such as coated pharmaceutical tablets, including branded tablets, and to methods of making and using the same.

### SUMMARY OF THE INVENTION

The present invention relates, inter alia, to a polish composition for coating pharmaceutical solid dosage forms such as pharmaceutical tablets. The polish composition comprises water, coating agent, and a film forming agent. The water is present in an amount of about 80-99% of the total weight of the polish composition. The film-forming agent is present in an amount of about 0.25-20% or about 1-20% of the total weight of the polish composition, and is, for example, a water soluble polymer such as water-soluble cellulose ethers, starches, disintegrants, dissolution aids, suspending agents, tablet binders, or combinations thereof. The coating agent is present in an amount of about 0.01-5% of the total weight of the polish composition, and is, for example, a wax, emollient, emulsifying agent, plasticizer, tablet/capsule lubricant, anti-caking agent, glidant, diluent, lubricant, solubilizing agent, opacifier, or colorant, or combinations thereof.

The present invention also relates to polished pharmaceutical solid dosage forms such as polished pharmaceutical tablets comprising polish components that form a polished exterior surface. The weight of the polish components (i.e., the polish solids, or the polish solids comprising the polished exterior surface) can be 5% or less of the total weight of said polished pharmaceutical tablet.

The present invention also provides processes of polishing pharmaceutical solid dosage forms such as pharmaceutical tablets. The processes comprise the steps of applying a polish composition to a heated base pharmaceutical solid dosage form such as a tablet; and drying the pharmaceutical solid dosage form such as the tablet with the polish composition. The water from the polish composition evaporates to produce the polished pharmaceutical tablet. The polish composition comprises water, coating agent, and a film forming agent. The water is present in an amount of about 80-99% of the total weight of the polish composition. The film-forming agent is present in an amount of about 0.25-20% or about 1-20% of the total weight of the polish composition, and can be a water soluble polymer such as water-soluble cellulose ethers, starches, disintegrants, dissolution aids, suspending agents, and tablet binders, or a combination thereof. The coating agent is present in an amount of about 0.01-5% of the total weight of the polish composition, and is a wax, emollient, emulsifying agent, plasticizer, tablet/capsule lubricant, anti-caking agent, glidant, diluent, lubricant, solubilizing agent, opacifier, or colorants, or a combination thereof.

### DETAILED DESCRIPTION OF EMBODIMENTS

In some embodiments, polish compositions for coating pharmaceutical solid dosage forms such as pharmaceutical tablets comprising water, a coating agent, and a film forming agent are provided. The water is present in an amount equal to about 80-99% of the total weight of the polish composition. The coating agent is present in an amount equal to about 0.01-5% of the total weight of the polish composition. The film-forming agent is present in an amount equal to about 0.25-20% of the total weight of the polish composition. Following application of the polish composition to pharmaceutical tablets, the tablets on which the polish composition applied are dried, allowing the water to evaporate and thereby depositing the non-volatile components (including the coating agent and the film-forming agent) of the composition on the tablets. The coating agent and the film-forming agent are generally cumulatively present in an amount equal to at least about 50% of total weight of residual material following drying, i.e. the dry weight of the composition.

The polish compositions are applied to the exterior of film or sugar coated tablets (or other film or sugar coated pharmaceutical solid dosage forms) in order to improve luster and/or slip. In some embodiments, the polish compositions of the present invention are applied to the exterior of film or sugar coated tablets to improve luster. In some embodiments, the improvement of luster can be measured visually or by a gloss meter. See e.g. US/20050061205. In some embodiments the polished tablets of the present invention visually have an improved luster comparing to the unpolished tablets. In some embodiments, the polish compositions of the present invention are applied to the exterior of film or sugar coated tablets to improve slip. In some embodiments, the improvement of slip can be measured by angle of repose. See e.g., US/20070216196, US/20080305170, and US/20090049935. In some embodiments, the angle of repose of an un-polished solid dosage form can be improved from about 350 or 400 to less than about 300, 250, 200, or 150 in an polished solid dosage form of the present invention. In some embodiments, the angle of repose of an un-polished solid dosage form can be reduced by about 5°, 10°, 15°, or 200 after application of the polish composition. In some embodiments, the angle of repose of an polished solid dosage form of the invention can be less than about 30°, 25°, 20°, 15°, or 10°. Tablets with improved slip are preferred in manufacturing and packaging processes, because reduced friction and adherence of tablets to machinery and equipment results in less production and process errors, such as mispackaging blister packs, which are costly and time consuming. Moreover, improved slip allows the consumer to more easily ingest polished tablets. The polished pharmaceutical tablets made by application of the polish compositions provided herein provide good slip properties, good brand adherence, and/or reduced tackiness when exposed to humidified conditions. In some embodiments, the brand adherence property of an polished solid dosage form of the invention is comparable to or better than that of an unpolished solid dosage form.

Pharmaceutical grade water is preferably used in the preparation of the polish composition. The water is present in an amount of about 80-99% of the total weight of the polish composition, in some embodiments preferably about 94-99%, in some embodiments preferably about 94-96%, and in some embodiments about 95%. In some embodiments, the water is present in an amount of about 90-99% of the total weight of the polish composition, in some embodiments, preferably about 94-99%, in some embodiments preferably about 94-96%, and in some embodiments about 95%. In some embodiments, the water is present in an amount of 90-99% of the total weight of the polish composition, in some embodiments preferably 94-99%, in some embodiments preferably 94-96%.

Examples of film-forming agents for use in the polish composition include water soluble polymers such as water-soluble cellulose ethers, starches, disintegrants, dissolution aids, suspending agents, and tablet binders. An example of tablet binder is polyvinylpyrrolidone (PVP, Povidone). Examples of water-soluble cellulose ethers for use in the polish composition include methyl cellulose, hydroxypropyl cellulose, hydroxyethyl cellulose, hydroxypropyl methyl cellulose, and hydroxyethyl methyl cellulose, and combinations thereof. In some embodiments the film-forming agent is hydroxypropyl methyl cellulose with a viscosity of about 3-15 cps, preferably about 3, 5, 6, or 15 cps. Examples of starches for use in the polish composition include natural starches (e.g., maize starch, rice starch, potato starch, wheat starch, and tapioca starch) and modified starches (for example, starches that have been modified with regard to molecular weight or branching; or starches that have been chemically modified to attach chemical functionality such as carboxy or hydroxyl groups). Examples of disintegrants for use in the polish composition include pregelatinized starch, sodium starch glycolate, croscarmellose sodium, crospovidone, starch, alginic acid, sodium alginate, clays (e.g. veegum or xanthan gum), and cellulose floc. Examples of dissolution aids for use in the polish composition include polyoxyethylene sorbitan fatty acid esters, polyethylene glycols, polyoxyethylene castor oil derivatives, sugar esters of fatty acids, and glycerides of fatty acids [for example. monoglycerides (such as glyceryl monostearate), diglycerides, and triglycerides. Examples of fatty acids include stearic acid and lauric acid]. Examples of suspending agents for use in the polish composition include cellulose (e.g., microcrystalline cellulose or methylcellulose) and povidone. Pharmaceutical grade material is preferably used in the preparation of the polish composition. The film forming agents are present in an amount of about 0.25-20% of the total weight of the polish composition. In some embodiments, the film forming agents are present in an amount of about 1-20% of the total weight of the polish composition, preferably about 4-5%, and in some embodiments about 4.95%. In some embodiments, the film forming agents are present in an amount of 1-20% of the total weight of the polish composition, preferably 4-5%.

Suitable coating agents may be hydrophobic. Examples of coating agents for use in the polish composition include waxes, emollients, emulsifying agents, plasticizers, tablet/capsule lubricants, anti-caking agents, glidants, diluents, lubricants, solubilizing agents, opacifiers, colorants, and combinations thereof. Pharmaceutical grade material is preferably used in the preparation of the polish composition. Waxes are typically suspended in the composition as particulates, rather than being molten or liquid. Wax is typically provided in particulates small enough to get through the sprayer holes found on equipment typically used for applying coatings to pharmaceutical tablets or other solid dosage forms. In some embodiments, wax is provided as a fine particulate, such as for example a particulate in which >90% will pass through a 120 mesh US standard screen. Examples of waxes for use in the polish composition include apple peel wax, avocado wax, bayberry wax, beeswax, candelilla wax, carnauba wax, jojoba wax, rice bran wax, lanolin wax, microcrystalline wax, mink wax, montan acid wax, montan wax, orange peel wax, ouricury wax, palm kernel wax, paraffin wax, and combinations thereof. In some embodiments, the plasticizers may be 0 to about 20% or 0 to about 10% of the amount of the coating agent present, in some embodiments, 0 to about 5% or about 5-10%. In some embodiments, the plasticizers may be 0 to about 20% or 0 to about 10% of the amount of the coating agent present, in some embodiments, 0 to about 5% or about 5-10%. Examples of plasticizers for use in the polish composition include propylene glycol, glycerin, glyceryl triacetate, polyethylene glycol, triethyl citrate, and dibuylsebecate.

Examples of emollients for use in the polish composition include glycerin and wax (such as avocado wax). Examples of emulsifying agents for use in the polish composition include one or more of metallic alkyl sulfate, quaternary ammonium compounds, salts of fatty acids, polyethylene glycol, and polypropylene glycol. Examples of tablet/capsule lubricants for use in the polish composition include one or more of stearic acid and metallic stearate (e.g. calcium stearate and magnesium stearate). Examples of anti-caking agents for use in the polish composition include sodium aluminosilicate, sodium silicate, silica (silicon dioxide), calcium silicate, and stearic acid. Examples of glidants for use in the polish composition include Talc, silica (silicon dioxide, such as colloidal silica), and starch. Examples of lubricants for use in the polish composition include one or more of stearic acid, stearic acid salts such as metallic stearate (e.g. calcium stearate and magnesium stearate), sodium stearyl fumarate, fatty acid, fatty alcohol, fatty acid ester (e.g. glyceryl monostearate), glyceryl behenate, mineral oil, vegetable oil, paraffin, leucine, silica (silicon dioxide), silicic acid, talc, propylene glycol fatty acid ester, polyethylene glycol, polypropylene glycol, polyalkylene glycol, or sodium chloride. Examples of solubilizing agents for use in the polish composition include metallic alkyl sulfate, quaternary ammonium compounds, glycerides of fatty acids [for example. monoglycerides (such as glyceryl monostearate), diglycerides, and triglycerides], and salts of fatty acids (e.g., calcium stearate and magnesium stearate). Examples of opacifiers for use in the polish composition include titanium dioxide, calcium carbonate, and iron oxides. Examples of colorants for use in the polish composition include titanium dioxide, calcium carbonate, and iron oxides.

In some embodiments, examples of the coating agents include glyceryl monostearate, silica (silicon dioxide), calcium stearate, magnesium stearate, talc, stearic acid, and combination thereof, alone or in combination with one or more waxes. In some embodiments, examples of the coating agents include glyceryl monostearate, magnesium stearate, talc, stearic acid, and combination thereof, alone or in combination with one or more waxes. The coating agent is present in an amount of about 0.01-5% of the total weight of the polish composition. In some embodiments, the coating agent is present in an amount of about 0.025-1% of the total weight of the polish composition. In some embodiments the coating agent is present in an amount of about 0.05% of the total weight of the polish composition. In some embodiments, the coating agent is present in an amount of 0.025-1% of the total weight of the polish composition.

Optionally, the polish composition may contain other ingredients. Preferably, 50% or more of the polish composition dry weight is attributable to the combination of weights of the coating agent and the film forming agent. In some embodiments, at least about 55%, 60%, 65%, 70%, 75%, 85%, 90%, 92%, 95%, 98%, or 99% of the polish composition dry weight is attributable to the combination of weights of the coating agent and the film forming agent. In some embodiments, at least 55%, 60%, 65%, 70%, 75%, 85%, 90%, 92%, 95%, 98%, or 99% of the polish composition dry weight is attributable to the combination of weights of the coating agent and the film forming agent.

The polish composition can be prepared as an emulsion, dispersion, or suspension, and can be used to coat pharmaceutical tablets and the like. The polish composition is typically prepared at room temperature. Typically, the coating agent is added to stirring water, followed by addition of the film-forming agent. The mixture is stirred until a uniform suspension is achieved. Additional water may be added to achieve a desired final volume and the suspension may be de-aerated. Polish composition may be applied to tablets, such as by spraying onto preheated tablets, in a sufficient amount to provide the desired luster and slip. For example, the composition may be applied using a standard spraying technique such as using a ¼ JAU spray nozzle with an E15 spray set up. Spraying pressures may preferably be about 30-60 psi. The bed temperature may be preferably about 40-45° C. In some embodiments, the polish composition is applied at a rate sufficient to allow the liquid on tablets to evaporate rapidly following the application. In some embodiments, the polish composition is applied at a rate sufficient to allow the liquid on tablets to completely evaporate rapidly following the application. In some embodiments, the polish composition is applied at a rate sufficient to allow the liquid on tablets to partially evaporate rapidly following the application. In some embodiments, the tablets are further dried after the application is completed. In some embodiments, the tablets are further dried after application is completed under a suitable condition (such as at a temperature of about 40-45° C.) until the weight of the tablets remains substantially constant (for example, the weight of the tablets changes less than about 0.1% in one hour at a suitable drying condition; i.e., until no additional substantial evaporation of liquid on tablets under the drying condition is observed). In some embodiments, the tablets are then dried completely after application is completed. In some embodiments, the polish solids (i.e., the polish components or the polish on the exterior of the polished tablets) comprise less than about 20%, 15%, 10%, 8%, 5%, 2%, 1%, 0.8%, 0.5%, 0.2%, or 0.1% of water by weight after the tablets are dried. In some embodiments, about 70-80% of the solid applied during spraying remains on tablets after the liquid is evaporated and the tablets are dry. The polish provides a very small amount of material on the exterior of the tablet to yield a smoother surface.

The amount of polish solids on a dry tablet is preferably about or less than about 5% of the total weight of the tablet; in some embodiments about or less than about 4%, in some embodiments about or less than about 3%, in some embodiments about or less than about 2.5%, in some embodiments about or less than about 2%, in some embodiments about or less than about 1.5%, in some embodiments about or less than about 1%, in some embodiments about or less than about 0.5%, in some embodiments between about 0.5% and about 5%, in some embodiments between about 0.5% and about 2.5%, and in some embodiments between about 1% and about 2%.

As used herein, the polish components of the polished pharmaceutical dosage form (such as a polished tablet) are also referred to as the polish solids or the polish on the exterior of the polished pharmaceutical dosage form (such as a polished tablet).

In some embodiments, the polished pharmaceutical solid dosage form (e.g. a tablet) comprises a base solid dosage form (e.g. a tablet) tablet that is a sugar coated pharmaceutical dosage form such as a sugar coated tablet (i.e., the polish components are applied over the sugar coated tablet). In some embodiments, the polished pharmaceutical solid dosage form (e.g. a tablet) comprises a base pharmaceutical solid dosage form (e.g. a tablet) that is a film coated pharmaceutical solid dosage form such as a film coated tablet (i.e., the polish components are applied over the film coated tablet). In some embodiments, the polished pharmaceutical tablet (or the base pharmaceutical tablet in the polished pharmaceutical tablet) comprises one or more active agents selected from the group consisting of: conjugated estrogens and conjugated estrogens combined with medroxyprogesterone acetate. In some embodiments, the polished pharmaceutical tablet (or the base pharmaceutical tablet in the polished pharmaceutical tablet) comprises one or more conjugated estrogens in doses selected from the group consisting of: about 0.3 mg/tablet, about 0.45 mg/tablet, about 0.625 mg/tablet, about 0.9 mg/tablet, and about 1.25 mg/tablet. In some embodiments, the polished pharmaceutical tablet (or the base pharmaceutical tablet in the polished pharmaceutical tablet) comprises one or more conjugated estrogens combined with medroxyprogesterone acetate in doses of conjugated estrogens/medroxyprogesterone acetate selected from the group consisting of: about 0.3 mg/1.5 mg per tablet, about 0.45 mg/1.5 mg per tablet, about 0.625 mg/2.5 mg per tablet, and about 0.625 mg/5 mg per tablet. In some embodiments, the one or more active agents in the polished pharmaceutical tablet are not present in the polish components of the polished pharmaceutical tablet.

As used herein, the term “about” in connection with a number (such as a percentage or other numbers) means that the number can be varied +/−10%. For example, “about 5%” can be a percentage of 4.5%-5.5% (e.g., 5.2%). For another example, “about 0.9 mg/tablet” refers to 0.81-0.99 mg/tablet (e.g., 0.88 mg/tablet). For yet another example, “about 0.45 mg/1.5 mg per tablet” refers to 0.405-0.495 mg/1.35-1.65 mg per tablet (e.g. 0.425 mg/1.55 mg per tablet).

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Example 1

### Polish Suspension Formulation

| Ingredient | Input/kg (g) | (% total weight) |
|---|---|---|
| Hypromellose 6 cps | 49.5 | (4.95%) |
| Carnauba Wax (≧90% thru 120 Mesh) | 0.5 | (0.05%) |
| Water | 950 | (95.0%) |

Polish Suspension Manufacturing Preparation:
1. To a suitable sized vessel equipped with stirring add the water at room temperature.
2. To Step 1 add the Carnauba Wax with stirring.
3. To Step 2 add the Hypromellose (i.e. Hydroxypropyl methylcellulose) with stirring.
4. Stir Step 3 until a uniform suspension is obtained by visual inspection.
5. QS (Adjust the final weight of the suspension) with water and de-aerate, if necessary.
6. Spray the target amount of Polish suspension onto a rotating pan load of preheated tablets until a suitable amount is applied to tablets using standard aqueous film coat procedures.

The polish formulation may be applied by a one step application procedure that provides good luster and tablet flow properties. The polished tablets (and/or the polish/polish components of the polished tablets) are less likely to have the problems identified with high humidity such as increased tackiness and/or decreased slip. The polished tablets (and/or the polish/polish components of the polished tablets) may absorb moisture without the problems identified with high humidity such as increased tackiness and decreased slip. This allows the tablets to be removed from the package easily by the patient and taken orally with no handling problems due to tackiness. Brand definition and adhesion properties are acceptable.

### Example 2

The following are examples of polish suspensions comprising a film forming agent and a coating agent in water that were prepared.

| Film-forming agent | Coating agent |
|---|---|
| Hydroxypropyl methylcellulose 2% (3 cps) | Calcium stearate (0.5%) |
| Hydroxypropyl methylcellulose 2% (3 cps) | Carnuba wax (0.5%) |
| Hydroxypropyl methylcellulose 2% (3 cps) | Carnuba wax (0.1%) |
| Hydroxypropyl methylcellulose 2% (3 cps) | Magnesium stearate (0.5%) |
| Hydroxypropyl methylcellulose 2% (3 cps) | GM stearate (0.5%) |
| Hydroxypropyl methylcellulose 2% (3 cps) | Stearic acid (0.5%) |
| Hydroxypropyl methylcellulose 2% (3 cps) | Silicon dioxide (0.25%) |
| Hydroxypropyl methylcellulose 2% (3 cps) | Talc (0.5%) |

As used herein, GM stearate refers to glyceryl monostearate

### Example 3

The following are examples of polish suspensions comprising a film forming agent and a coating agent in water that were prepared and used for polishing. The angle of repose of the examples of the polished tablets (and the unpolished tablets) are also listed.

| Example | | | angle of |
|---|---|---|---|
| numbers | Film-forming agent | Coating agent | repose |
| 3a (un-polished | — | — | 36° |
| tablets: green, | | | |
| oval biconvex | | | |
| coated tablets) | | | |
| 3b | Hydroxypropyl | Carnuba wax | 14° |
| | methylcellulose 2% (3 cps) | (0.5%) | |
| 3c | Hydroxypropyl | Carnuba wax | 16° |
| | methylcellulose 2% (3 cps) | (0.1%) | |
| 3d | Hydroxypropyl | Magnesium | 23° |
| | methylcellulose 2% (3 cps) | stearate (0.5%) | |
| 3e | Hydroxypropyl | Calcium stearate | 19° |
| | methylcellulose 2% (3 cps) | (0.5%) | |
| 3f | Hydroxypropyl | GM | 16° |
| | methylcellulose 2% (3 cps) | stearate (0.5%) | |
| 3g | Hydroxypropyl | Stearic | 18° |
| | methylcellulose 2% (3 cps) | acid (0.5%) | |
| 3h | Hydroxypropyl | Silicon dioxide | 30° |
| | methylcellulose 2% (3 cps) | (0.25%) | |
| 3i | Hydroxypropyl | Talc (0.5%) | 29° |
| | methylcellulose 2% (3 cps) | | |

Although this application refers specifically to tablets, those skilled in the art would recognize that the polish composition and methods of its application described herein are applicable to other solid dosage forms including, but not limited to, caplets. Further, the polish composition and methods of its application are suitable for use with various tableting techniques such as dry and wet granulations, compression, etc.

Various modifications of the invention, in addition to those described herein, will be apparent to those skilled in the art from the foregoing description. Such modifications are also intended to fall within the scope of the appended claims. Each reference cited in the present application is herein incorporated by reference in its entirety.

What is claimed is:

1. A polish composition for coating a pharmaceutical solid dosage form comprising water, coating agent, and a film forming agent; wherein:
said water is present in an amount of about 80-99% of the total weight of the polish composition;
said film-forming agent is present in an amount of about 0.25-20% of the total weight of the polish composition and is a water soluble polymer selected from the group consisting of: water-soluble cellulose ethers, starches, disintegrants, dissolution aids, suspending agents, tablet binders, and combinations thereof;
said coating agent is present in an amount of about 0.01-5% of the total weight of the polish composition and is selected from the group consisting of: waxes, emollients, emulsifying agents, plasticizers, tablet/capsule lubricants, anti-caking agents, glidants, diluents, lubricants, solubilizing agents, opacifiers, colorants, and combinations thereof.

2. The polish composition of claim 1 wherein said film-forming agent is present in an amount of about 1-20% of the total weight of the polish composition.

3. The polish composition of claim 1 wherein said film-forming agent is selected from the group consisting of: polyvinylpyrrolidone, methylcellulose, hydroxypropyl cellulose, hydroxyethyl cellulose, hydroxypropyl methylcellulose, hydroxyethyl methylcellulose, and combinations thereof.

4. The polish composition of claim 3 wherein said water-soluble cellulose ether is hydroxypropyl methylcellulose.

5. The polish composition of claim 3 wherein said water-soluble cellulose ether is hydroxypropyl methylcellulose having a viscosity of about 3-15 cps.

6. The polish composition of claim 1 wherein said coating agent is selected from the group consisting of: apple peel wax, avocado wax, bayberry wax, beeswax, candelilla wax, carnauba wax, jojoba wax, rice bran wax, lanolin wax, microcrystalline wax, mink wax, montan acid wax, montan wax, orange peel wax, ouricury wax, palm kernel wax, paraffin wax, glyceryl monostearate, magnesium stearate, talc, stearic acid, propylene glycol, glycerin, glyceryl triacetate, polyethylene glycol, triethyl citrate, dibuylsebecate, and combinations thereof.

7. The polish composition of claim 1 wherein said coating agent is carnauba wax.

8. The polish composition of claim 1 wherein said water is present in an amount of about 90-99% of the total weight of the polish composition.

9. The polish composition of claim 1 wherein said water is present in an amount of about 94-96% of the total weight of the polish composition.

10. The polish composition of claim 1 wherein said film-forming agent is present in an amount of about 4-5% of the total weight of the polish composition.

11. The polish composition of claim 1 wherein said coating agent is present in an amount of about 0.025-1% of the total weight of the polish composition.

12. The polish composition of claim 1 wherein said coating agent is present in an amount of about 0.05% of the total weight of the polish composition.

13. The polish composition of claim 1 wherein the solid dosage form is a tablet.

14. A polished pharmaceutical solid dosage form comprising a base pharmaceutical solid dosage form; and polish components forming a polished exterior surface of polished pharmaceutical solid dosage form, wherein:
the polish components comprise a filming-forming agent and a coating agent;
the film-forming agent comprises a water soluble polymer selected from the group consisting of: water-soluble cellulose ethers, starches, disintegrants, dissolution aids, suspending agents, tablet binders, and combinations thereof; and
the coating agent is selected from the group consisting of: waxes, emollients, emulsifying agents, plasticizers, tablet/capsule lubricants, anti-caking agents, glidants, diluents, lubricants, solubilizing agents, opacifiers, colorants, and combinations thereof.

15. A polished pharmaceutical solid dosage form comprising a base pharmaceutical solid dosage form; and polish components forming a polished exterior surface of the polished pharmaceutical solid dosage form, wherein:
the polish components comprise a filming-forming agent and a coating agent;
the film-forming agent comprises a water soluble polymer selected from the group consisting of: water-soluble cellulose ethers, starches, disintegrants, dissolution aids, suspending agents, tablet binders, and combinations thereof;
the coating agent is selected from the group consisting of: waxes, emollients, emulsifying agents, plasticizers, tablet/capsule lubricants, anti-caking agents, glidants, diluents, lubricants, solubilizing agents, opacifiers, colorants, and combinations thereof; and
the polished pharmaceutical solid dosage form is prepared using a polish composition of claim 1.

16. The polished pharmaceutical solid dosage form of claim 14 wherein the weight of the polish components is about 5% or less of the total weight of said polished pharmaceutical solid dosage form.

17. The polished pharmaceutical solid dosage form of claim 14 wherein the weight of the polish components is about 3% or less of the total weight of said polished pharmaceutical solid dosage form.

18. The polished pharmaceutical solid dosage form of claim 14 wherein the weight of the polish components is about 2% or less of the total weight of said polished pharmaceutical solid dosage form.

19. The polished pharmaceutical solid dosage form of claim 14 wherein the weight of the polish components is about 1-2% of the total weight of said polished pharmaceutical solid dosage form.

20. The polished pharmaceutical solid dosage form of claim 14 wherein said base pharmaceutical solid dosage form is a sugar coated pharmaceutical solid dosage form.

21. The polished pharmaceutical solid dosage form of claim 14 wherein said base pharmaceutical solid dosage form is a film coated pharmaceutical solid dosage form.

22. The polished pharmaceutical solid dosage form of claim 14 wherein said base pharmaceutical solid dosage form comprises one or more active agents selected from the group consisting of: conjugated estrogens and conjugated estrogens combined with medroxyprogesterone acetate.

23. The polished pharmaceutical solid dosage form of claim 14 wherein said base pharmaceutical solid dosage form comprises one or more conjugated estrogens in doses selected from the group consisting of: about 0.3 mg/tablet, about 0.45 mg/tablet, about 0.625 mg/tablet, about 0.9 mg/tablet, and about 1.25 mg/tablet.

24. The polished pharmaceutical solid dosage form of claim 14 wherein said base pharmaceutical solid dosage form comprises one or more conjugated estrogens combined with medroxyprogesterone acetate in doses of conjugated estrogens/medroxyprogesterone acetate selected from the group consisting of: about 0.3 mg/1.5 mg per tablet, about 0.45 mg/1.5 mg per tablet, about 0.625 mg/2.5 mg per tablet, and about 0.625 mg/5 mg per tablet.

25. The polished pharmaceutical solid dosage form of claim 14 wherein the solid dosage form is a tablet.

26. A process of polishing a pharmaceutical solid dosage form comprising the steps of:
applying a polish composition of claim 1 to a heated base pharmaceutical solid dosage form; and
drying said base pharmaceutical solid dosage form with the polish composition; wherein the water from the polish composition evaporates to produce the polished pharmaceutical solid dosage form.

27. The process of claim 26 wherein the polish components of the polished pharmaceutical solid dosage form comprise 5% or less of the total weight of the polished pharmaceutical solid dosage form.

28. The process of claim 26 wherein the solid dosage form is a tablet.
